(19) United States
(12) Patent Application Publication
(10) Pub. No.: US 2023/0414653 A1
(43) Pub. Date: Dec. 28, 2023

(54) TOPICAL CHLORINE DIOXIDE TREATMENT FOR MAMMALIAN TEATS

(71) Applicant: ECOLAB USA INC., St. Paul, MN (US)

(72) Inventors: Daniel Alexander SIEBOLD, Leichlingen (DE); Helen van den BROEK, Brueggen (DE); Michael SCHNEIDER, Grevenbroich (DE)

(21) Appl. No.: 18/340,484

(22) Filed: Jun. 23, 2023

Related U.S. Application Data
(60) Provisional application No. 63/355,417, filed on Jun. 24, 2022.

(51) Int. Cl. A61K 33/20; A61K 9/00; A61K 45/06; A61P 31/04; C01B 11/02

(52) U.S. Cl. CPC A61K 33/20; A61K 9/0017; A61K 45/06; A61P 31/04; C01B 11/024

(57) ABSTRACT

Chlorine dioxide compositions and methods of using the chlorine dioxide compositions on animal skin are disclosed. The chlorine dioxide compositions are formed by mixing an acidic composition and a chlorite salt composition together. The acidic composition includes an acid and an aldehyde.

### CROSS REFERENCE TO RELATED APPLICATIONS

This application is being filed on Jun. 23, 2023, as a PCT International application and claims priority to U.S. Provisional Application No. 63/355,417, filed on Jun. 24, 2022 entitled “Topical Chlorine Dioxide Treatment for Mammalian Teats”, which is incorporated by reference herein in its entirety.

### BACKGROUND

The skin and teats of milking animals are subjected to a number of stressors including extreme temperature and weather conditions, vacuum pressure from automated milking equipment, and microorganisms encountered as animals walk through the fields and barns. If microorganisms infect the mammary glands of milking animals, the infection can lead to mastitis. While mastitis is treatable using antibiotics, milk from the infected animal cannot be sold until the infection is over and the antibiotic has passed out of the milk. This can take several days.

Topical treatments for mammalian teats are used to prevent mastitis infections in milking animals, maintain or protect the integrity of the skin around the teat, and protect against microorganisms encountered as animals walk in the field or barn. Chlorine dioxide is used in such topical treatments. Chlorine dioxide is a desirable antimicrobial because it has broad spectrum activity against different kinds of microorganisms, it achieves a significant log reduction against those microorganisms, and it is effective in the presence of a high soil load. But, chlorine dioxide is a gas and not stable over a longer period of time.

Chlorine dioxide topical treatments are formed from a two-part composition where sodium chlorite is combined with an acid to form the chlorine dioxide. Once the chlorine dioxide is formed, it is not stable indefinitely. It will gas off or react with other ingredients in the formulation. In order to ensure that the chlorine dioxide can be used for as long as possible, the dairy farmer usually combines the sodium chlorite and acid components to form the chlorine dioxide. The reaction time between the sodium chlorite and acid components is typically slow. The reaction rate can be increased by increasing the concentrations of the sodium chlorite and acid components but as the reaction progresses, the higher concentration of components will eventually generate unsafe quantities of chlorine dioxide. Accordingly, when a dairy farmer is using chlorine dioxide topical treatments, the farmer faces multiple problems: the farmer must mix the chemistry together shortly before use in order to ensure a long shelf life, the time between mixing and using can be unacceptably long in order for a sufficient amount of chlorine dioxide to form, and once the chlorine dioxide is formed, it must be used quickly. It is against this background that the present disclosure is made.

### SUMMARY

In some aspects, the present disclosure relates to a kit for forming a chlorine dioxide composition. The kit includes an acidic composition with an acid, an aldehyde, and water. The kit also includes a chlorite composition comprising a chlorite salt and water. When the acidic composition and the chlorite composition are combined, they form a chlorine dioxide composition.

In some aspects, the present disclosure relates to a chlorine dioxide generation system. The system includes an acidic composition with an acid and an aldehyde. The system also includes a chlorite composition with a chlorite salt.

In some aspects, the present disclosure relates to a method of generating a chlorine dioxide composition comprising mixing an acidic composition and a chlorite composition together to form a chlorine dioxide composition. The acidic composition includes an acid and an aldehyde. The chlorite composition includes a chlorite salt.

In some aspects, the present disclosure relates to a method of reducing microorganisms on animal skin. According to this method, an acidic composition and a chlorite composition are mixed together to form a chlorine dioxide composition. The acidic composition includes an acid and an aldehyde. The chlorite composition includes a chlorite salt. When the acidic composition and chlorite composition are mixed together, they form chlorine dioxide in a chlorine dioxide composition. The chlorine dioxide composition is applied to animal skin. In some aspects, the chlorine dioxide composition is a pre-milking composition. In some aspects, the chlorine dioxide composition is a post-milking composition.

### DETAILED DESCRIPTION

The present disclosure relates to chlorine dioxide compositions for use on the skin and teats of milking animals and to the starting compositions, kits, and systems for making the chlorine dioxide compositions, methods of generating the chlorine dioxide compositions, and methods of using the chlorine dioxide compositions. The chlorine dioxide compositions can be used pre- and post-milking to prevent or treat mastitis infections. The desired properties for pre- and post-milking compositions may be different. For example, the objective with pre-milking compositions is often to clean the animal skin in preparation for milking. Pre-milking compositions encounter a high soil load and must be able to remove the soil and reduce any microorganisms in the presence of the soil load. Pre-milking compositions also cannot interfere with the milk, the milking equipment, or the milking process. Post-milking compositions generally encounter animal skin that has just been milked. The skin should be clean and the objective with the post-milking composition is supporting the skin health after milking and reducing microorganisms as the animal leaves the barn and goes back to the field. Post-milking compositions may contain additives to help the composition stay on the animal skin or support skin health. Both pre- and post-milking chlorine dioxide compositions can be formulated to provide additional benefits such as improved skin health or provide a visual indicator that the product has been applied or an animal has been milked.

### Chlorine Dioxide Compositions

Chlorine dioxide compositions are typically formed from two starting compositions. Before using the chlorine dioxide composition in a pre-milking application, a dairy farmer will typically combine the two compositions together and then have to wait from 15 minutes up to 1 hour for the chlorine dioxide to be formed. Once the chlorine dioxide is formed, the pre-milking composition typically needs to be consumed or disposed of within 8 hours. For post-milking compositions, the chlorine dioxide formation is the same (from 15 minutes up to 1 hour) but the compositions can be used for up to 26 days. Post-milking compositions can be used for a longer period of time because they are used on animal skin that is cleaner than the pre-milking animal skin. The disclosed chlorine dioxide compositions provide faster chlorine dioxide generation and longer chlorine dioxide stability.

The disclosed chlorine dioxide compositions are formed from the reaction of two starting compositions: an acidic composition and a chlorite salt composition. The acidic composition includes an acid and an aldehyde. The aldehyde provides two benefits. First, the aldehyde reacts with the chlorite salt to form chlorine dioxide. This reaction proceeds faster than the reaction between the acid and the chlorite salt and allows a dairy farmer to combine the acidic composition and the chlorite salt composition together and use the resulting chlorine dioxide compositions shortly after mixing. Because the aldehyde/chlorite reaction proceeds faster than the acid/chlorite reaction, the composition achieves the necessary level of chlorine dioxide much faster than in compositions with only the acid and not the aldehyde. Second, once the aldehyde is consumed, the acid remains available to continue to react with the chlorite salt and produce chlorine dioxide. This results in longer chlorine dioxide generation and longer chlorine dioxide availability and shelf life.

The reaction between the aldehyde and chlorite is completed in less than a minute at room temperature and produces 2 moles of chlorine dioxide for every 1 mole of the aldehyde. In some embodiments, the aldehyde is only present in the acidic composition and is immediately converted into a carboxylic acid once it is mixed with the chlorite salt so that there is no, or substantially no aldehyde in the chlorine dioxide composition. In some embodiments, the aldehyde is present in the chlorine dioxide composition at concentrations of less than 50 ppm, less than 10 ppm, less than 5 ppm, less than 1 ppm, or is absent 1 minute after mixing the acidic composition and the chlorite composition.

The reaction between the acid and the chlorite is typically slow to proceed compared to the aldehyde reaction. This reaction starts immediately but takes several days to complete at room temperature. The reaction speed can be increased by lowering pH or by increasing the concentration of the acid, the chlorite salt, or both. This results in faster chlorine dioxide generation, but after a few hours, generates unsafe levels of chlorine dioxide.

When the aldehyde and the acid are both included in the acid composition, the aldehyde and acid reactions with the chlorite salt overlap. The reaction with aldehyde happens within less than a minute, while the acid/chlorite reaction takes much longer. This is beneficial because the chlorine dioxide generated from the aldehyde reaction is available immediately to provide an antimicrobial benefit. The chlorine dioxide generated from the acid reaction takes longer to produce and thus maintains the antimicrobial properties of the composition for a longer period of time.

The chlorine dioxide compositions described herein generate chlorine dioxide that includes residual chlorite in the composition. As described above, the remaining chlorite in the composition continues to react with the acid in the solution, even after chloride dioxide is initially produced from the reaction of chlorite and the aldehyde. The chlorine dioxide compositions, which include unreacted chlorite that will subsequently react with the aldehyde, may be used as pre- and post-milking compositions applied to the surface of animal skin.

In some embodiments, the chlorine dioxide is used as a pre-milking composition. In Europe, the pre-milking composition must demonstrate a 5-log reduction (EN1656) and a 4-log reduction (EN1657) against target microorganisms within 60 seconds at 30° C., using an 80% dilution of the composition under dirty conditions. The pre-milking composition should also meet any requirements for efficacy tests on skin such as the Phase 2 Step 2 efficacy tests or EN17422 test on artificial skin.

In some embodiments, the chlorine dioxide is used as a post-milking composition and must demonstrate a 4-log reduction (EN1656) and a 3-log reduction (EN1657) against target microorganisms within 5 minutes at 30° C., using an 80% dilution in the presence of 1% skim milk.

In some embodiments, within 5 minutes after mixing the acidic composition and chlorite composition together to form the pre-milking chlorine dioxide composition, the resulting pre-milking chlorine dioxide composition achieves a 3- to 5-log reduction, a 3-log reduction, a 4-log reduction, or a 5-log reduction against E. coli, S. aureus, S. uberis, or Candida albicans within 60 seconds at 30° C. In some embodiments, the pre-milking composition achieves the desired log reduction within 1 minute after mixing, within 30 seconds after mixing, or within 10 seconds after mixing the acidic and chlorite compositions together.

In some embodiments, within 5 minutes after mixing the acidic composition and chlorite composition together to form the post-milking chlorine dioxide composition, the resulting post-milking chlorine dioxide composition achieves a 3- to 5-log reduction, a 3-log reduction, a 4-log reduction, or a 5-log reduction against E. coli or S. aureus within 5 minutes at 30° C. In some embodiments, the post-milking composition achieves the desired log reduction within 1 minute after mixing, within 30 seconds after mixing, or within 10 seconds after mixing the acidic and chlorite compositions together.

Once the aldehyde and chlorite have reacted together to form chlorine dioxide, the acid remains available to react with the remaining chlorite and provide ongoing chlorine dioxide generation. In some embodiments, the chlorine dioxide compositions achieve the desired log reduction (e.g., 3-log, 4-log, 5-log, 3-5 log) against target microorganisms such as E. coli, S. aureus, S. uberis, or C. albicans for up to 10 days, up to 14 days, up to 15 days, up to 20 days, up to 21 days, up to 25 days, up to 30 days, up to 35 days, up to 40 days, up to 45 days, up to 50 days, up to 55 days, or up to 60 days, days, 20-60 days, 30-60 days, 40-60 days, or 50-60 days after the acidic and chlorite compositions are combined at 30° C. and within 5 minutes, 4 minutes, 3 minutes, 2 minutes, 60 seconds, or 30 seconds of contact. This is true for either a pre-milking composition, a post-milking composition, or both.

Within 5 minutes after the acidic composition and chlorite composition are combined, the chlorine dioxide compositions include chlorine dioxide, residual chlorite salt and acid from the starting compositions, and optional additional ingredients. As the reaction proceeds, the chlorine dioxide concentration ranges from about 50 ppm to about 3000 ppm, from about 60 ppm to about 2000 ppm, from about 75 ppm to about 1000 ppm, from about 100 ppm to about 1000 pm, from about 250 ppm to about 1500 ppm, from about 400 ppm to about 1000 ppm, from about 200 ppm to about 600 ppm, or from about 75 ppm to about 500 ppm. The chlorine dioxide concentration should be within the desired range from about 5 minutes after the acidic composition and the chlorite composition are combined and for up to 10 days, 14 days, 15 days, 20 days, 21 days, 25 days, 30 days, 35 days, 40 days, 45 days, 50 days, 55 days, or 60 days, or 10-days, 20-60 days, 30-60 days, 40-60 days, or 50-60 days after combining the acidic composition and the chlorite composition. The reaction between the chlorite salt and acid may continue for minutes, hours, days, or weeks after combining the acidic composition and the chlorite composition.

Over time, the aldehyde concentration and the acid concentration in the chlorine dioxide compositions will decrease from their concentrations in the acidic composition as they are consumed to form the chlorine dioxide. The aldehyde concentration in the chlorine dioxide compositions is less than 50 ppm, less than 10 ppm, less than 5 ppm, less than 1 ppm, or is absent 1 minute after mixing the acidic composition and the chlorite composition. The concentration of the chlorite salt in the chlorine dioxide composition is preferably 0.04 to about 12.5 wt. %, from about 0.1 to about 10 wt. %, or from about 0.2 to about 9 wt. %. The concentration of the acid in the chlorine dioxide composition is preferably 0.1 to 10 wt. %, 0.1 to 5 wt. %, or 0.1 to 2 wt. %. The pH of the chlorine dioxide composition is preferably in the range of 2 to 7, 2 to 5, or 2 to 4.

The chlorine dioxide composition can also include additives such as diluents, pH adjusters, buffers, surfactants, emollients, moisturizers, film formers, foaming agents, thickeners, dyes, preservatives, additional antimicrobial agents, and mixtures thereof.

### The Acidic Composition

The acidic composition includes an acid, an aldehyde, and optional additional ingredients.

The acid may be an organic or inorganic acid. Exemplary acids include sulfamic acid, phosphoric acid, alkane sulfamic acid, formic acid, acetic acid, hydroxyacetic acid, citric acid, tartaric acid, lactic acid, glycolic acid, adipic acid, succinic acid, propionic acid, malic acid, cycloalkane sulfonic acids, C2 to C6 alphahydroxy carboxylic acids, C7 to C11 carboxylic acids, hydrocarbon sulfonic acids, and mixtures thereof. In some embodiments, the acid is preferably citric acid, acetic acid, lactic acid, glycolic acid, and mixtures thereof.

The acid is preferably present in the acidic composition in an amount from about to 10 wt. %, from about 0.1 to 5 wt. %, or from about 0.1 to 2 wt. %. The acid may be present in the acidic composition in an amount from about 0.1 to 9 wt. %, from about 0.1 to 8 wt. %, from about 0.1 to about 7 wt. %, from about 0.1 to about 6 wt. %, from about to about 4 wt. %, from about 0.1 to about 3 wt. %, from about 0.5 wt. % to about 10 wt. %, from about 1 wt. % to about 10 wt. %, from about 2 wt. % to about 10 wt. %, from about 3 wt. % to about 10 wt. %, from about 4 wt. % to about 10 wt. %, or from about 5 wt. % to about 10 wt. %.

The pH of the acidic composition preferably ranges from about 2 to about 7, from about 2 to about 6, from about 2 to about 4, or from about 2 to about 3.

The aldehyde can be any aldehyde that has the R-COH aldehyde functional group. Exemplary aldehydes include glutaraldehyde, formaldehyde, glyoxylic acid, glyoxal, succinaldehyde, adipaldehyde, and mixtures thereof.

The aldehyde is preferably present in the acidic composition in an amount from about 50 ppm to about 3000 ppm, from about 60 ppm to about 2000 ppm, from about ppm to about 1000 ppm, from about 400 ppm to about 1000 ppm, from about 200 ppm to about 600 ppm, or from about 75 ppm to about 500 ppm. In some examples, the aldehyde is present in the acidic composition in an amount less than 20,000 ppm, less than 10,000 ppm, less than 5,000 ppm, less than 4,000 ppm, less than 3,000 ppm, or less than 2,000 ppm.

The acidic composition can optionally include additional additives including diluents, pH adjusters, buffers, surfactants, emollients, moisturizers, film formers, foaming agents, thickeners, dyes, preservatives, additional antimicrobial agents, and mixtures thereof. In some embodiments, the additional additives are selected to be stable within the acidic composition or within the chlorine dioxide composition and not degraded by the acidic composition or the chlorine dioxide composition.

In the acidic composition, the composition may optionally include water as a diluent. Water may be present in the acidic composition in an amount from about 80 to about 99.99 wt. %, from about 90 to about 99.99 wt. %, or from about 95 to about 99.99 wt. %. In some embodiments, the diluent may include propylene glycol in concentrations up to about 10 wt. %, 0.1 to 10 wt. %, 0.1 to 5 wt. %, 0.1 to 2 wt. %, 2 wt. % to 5 wt. %, or 8 wt. % to 10 wt. %. Propylene glycol may be included as the only diluent or may be included with water.

The acidic composition may optionally include one or more pH adjusters or buffers to formulate to a desired pH. Exemplary pH adjusters include sodium hydroxide, potassium hydroxide, monoethanolamine, diethanolamine, triethanolamine, sodium carbonate, sodium bicarbonate, potassium carbonate, and potassium bicarbonate. Preferred pH adjusters are sodium hydroxide and potassium hydroxide. The pH adjusters can be present in an amount from about 0.001 to about 5 wt. %, from about 0.01 to about 2 wt. %, or from about 0.1 to about 1 wt. %.

The acidic composition may optionally include one or more surfactants. Exemplary surfactants include nonionic, anionic, cationic, or amphoteric surfactants. Preferred surfactants do not react with or degrade in the presence of the chlorine dioxide and are compatible with animal skin and for use as a teat dip. Preferred surfactants are also approved for food contact. Alkyl polyglucosides, anionic alcohol sulfates, sodium lauryl sulfate, sodium lauryl ether sulfate, and mixtures thereof are examples of preferred surfactants. The surfactant can be present in the acidic composition in amounts from about 0.01 to about 10.00 wt. %, from about 0.05 to about 5.00 wt. %, from about 0.1 to about 4 wt. %, from about 1 to about 5 wt. %, from about 2 to about 8 wt. %, from about 3 to about 7 wt. %, from about 5 to about 10 wt. %.

The acidic composition may optionally include one or more emollients, moisturizers, or humectants. The emollients, moisturizers, and humectants help ensure skin integrity, protect the skin, improve skin health and integrity, promote skin elasticity, or promote healing of dry or cracked skin. The skin is an important barrier to stop microorganisms from infecting the mammary gland and causing mastitis. Ensuring that the animal skin is elastic and free of cracks keeps the skin barrier strong. Exemplary emollients, moisturizers, and humectants include polyhydric alcohols such as glycerin, sorbitol, mannitol, and propylene glycol and its homopolymers; fatty acid esters of simple monohydril alcohols including isopropyl palmitate or isopropyl myristate and similar esters; polyol esters of fatty acids; C6 to C18 or C8 to C16 fatty acids and preferably saturated fatty acids, and ethoxylated lanolins, and other natural sourced derivatives such as aloe. Glycerine and sorbitol are preferred emollients, moisturizers, and humectants. Preferred emollients, moisturizers, and humectants do not react with the chlorine dioxide. Emollients, moisturizers, and humectants can be present in the acidic composition in amounts from about 0.5 to about 30 wt. %, from about 1 to about 20 wt. %, or from about 4 to about 15 wt. %.

The acidic composition may optionally include a foaming agent. In some embodiments, a foaming agent is useful in either a pre- or post-milking composition. Foaming is beneficial because it provides a visual indicator that product has been applied and where it has been applied. Dyes are not preferred in pre-milking compositions because there is a chance that the dyes could get into the milk. It is preferred to avoid getting dyes into the milk to the extent that the dyes are not approved for food contact and to avoid coloring the milk. Foaming is also beneficial because the foam increases the contact time with the skin and allows the chlorine dioxide to contact the skin for a longer time in order to achieve the desired microorganism reduction. Foaming is also typically achieved using surfactants. Those surfactants also help clean the skin and remove soils. Exemplary foaming agents include the surfactants described above. A foaming agent can be present in the acidic composition in amounts from about 0.01 to about 10.00 wt. %, from about 0.05 to about 5.00 wt. %, or from about 0.1 to about 4 wt. %.

The acidic composition may optionally include a thickener. Thickeners are beneficial because a thicker composition clings to the animal skin for longer and provides the chlorine dioxide more time to reduce microorganisms. Thickeners may or may not be film forming. Exemplary thickeners include silicas, silicates, gums, clays, polysaccharides, celluloses, water soluble polymers, and mixtures thereof. More specific examples include colloidal magnesium aluminum silicate (VEEGUM®), colloidal clays (Bentonites), or silicas (CAB-O-SILS®) which have been fumed or precipitated to create particles with large surface to size ratios. Exemplary natural hydrogel thickeners are primarily vegetable derived exudates such as tragacanth, karaya, and acacia gums; and extractives such as carrageenan, locust bean gum, guar gum and pectin; or, pure culture fermentation products such as xanthan gum. Chemically, all of these materials are salts of complex anionic polysaccharides. Synthetic natural-based thickeners having application are cellulosic derivatives wherein the free hydroxyl groups on the linear anhydro-glucose polymers have been etherified or esterified to give a family of substances which dissolve in water and give viscous solutions. This group of materials includes the alkyl and hydroxyllalkycelluloses, specifically methylcellulose, hydroxyethylmethylcellulose, hydroxypropylmethylcellulose, hydroxybutylmethycellulose, hydroxyethylcellulose, ethylhydroxyethylcellulose, hydroxypropylcellulose, and carboxymethylcellulose. Synthetic petroleum-based water soluble polymers are prepared by direct polymerization of suitable monomers of which polyvinylpyrrolidone, polyvinylmethylether, polyacrylic acid and polymethacrylic acid, polyacrylamide, polyethylene oxide, and polyethyleneimine are representative.

Preferred thickeners are those which are extremely pseudoplastic (non-Newtonian, rapid relaxation), tend not to develop a rigid three-dimensional structure from interpolymer interactions, have a low or negligible viscoelastic character and possess a high gel strength. The properties create compositions which have a smooth flowing appearance, are easy to pour and apply onto animal skin and teats, coat uniformly without forming muscilage streamers, and remain firmly in place without significant sag. Preferred thickeners include xanthan gum. The thickener can be present in the acidic composition in amounts from about 0.01 to about 5.00 wt. %, from about 0.05 to about 2 wt. %, or from about 0.1 to about 1 wt. %. The viscosity of the compositions with the thickeners should be up to 3000 mPas and preferably up to 2000 mPas when measured with a Brookfield viscometer spindle 2 with 12 rpm at 20° C. The composition is preferably mixable without too much effort.

The acidic composition may optionally include film formers. Film formers are useful if the chlorine dioxide composition is intended to remain on the animal skin or teat for a period of time. The film formers help the composition dry into a film that allows the chlorine dioxide to remain in contact with the animal skin for a period of time and provide longer contact with microorganisms. The film formers also form a barrier. This barrier can protect the skin from the environmental elements such as cold and wind. The barrier can also reduce microorganisms that the animal encounters as it walks around the field, barn, or pen. After milking, it may be beneficial to apply a post-milking spray or teat dip that stays on the animal either until the next milking or until it wears off. Exemplary film formers include the thickeners above that dry to form barriers and can include silicas, silicates, gums, clays, polysaccharides, celluloses, water soluble polymers, and mixtures thereof. Polyvinyl alcohol is a preferred film former. Exemplary polyvinyl alcohol polymers are those with a degree of hydrolysis greater than 92%, preferably greater than 98%, most preferably greater than 98.5%, and has a molecular weight (Mn) that falls in the range of between about 15,000 and 100,000, but preferably between 40,000 and 70,000 corresponding to a solution viscosity (4 wt. % aqueous solution measured in centipoise (cP) at 20° C. by Hoeppler falling ball method) of 12-55 cP and 12-25 cP respectively. Film formers can be present in the acidic composition in amounts from about 0.1 to about 7 wt. %, from about 0.5 to about 6 wt. %, or from about 1 to about 5 wt. %.

The acidic composition may optionally include a dye. Adding a color to the acidic composition and the resulting chlorine dioxide composition aids the dairy farmer with ensuring that the chlorine dioxide composition has been applied to the entire teat area. When applied as part of a post-milking composition, especially one that includes a film former and remains on the animal for a period of time, the dye is an indicator of which animals have been milked and which animals need to be milked. Exemplary dyes are approved for food contact and are not oxidized by chlorine dioxide and include food dyes, azo dyes, Direct Yellow 28, Pigment Green 7, and phthalocyanine-based dyes. The dye can be present in the acidic composition in an amount from about 0.001 to about 2 wt. %, from about 0.01 to about 1 wt. %, or from about 0.05 to about 0.5 wt. %. In some embodments, the pre-milking composition is free of a dye. In some embodiments, the post-milking composition is free of a dye.

The acidic composition may optionally include a preservative. Because the chlorine dioxide is formed when the acidic composition and the chlorite composition are combined, it may be beneficial to include a preservative in the acidic composition to prevent microorganisms from growing in that composition before it is mixed with the chlorite composition. Exemplary preservatives include benzoate, chlorocresol, methylisothiazolinone (MIT), benzisothiazolinone (BIT), 5-chloro-2-methyl-4-isothiazolin-3-one (CIT), and methylchloroisothiazolinone and methylisothiazolinone (CIT/MIT). The preservative can be included in an amount from about 10 ppm to about ppm, from about 100 ppm to about 2000 ppm, or from about 10 ppm to about 400 ppm.

### The Chlorite Composition

The chlorite composition includes a chlorite salt. Exemplary chlorite salts include alkali metal chlorites such as sodium chlorite and potassium chlorite, and mixtures thereof. A preferred chlorite salt is sodium chlorite. The chlorite salt is present in the chlorite composition in an amount from about 0.04 to about 12.5 wt. %, from about 0.01 to about 10 wt. %, from about 0.1 to about 10 wt. %, from about 0.2 to about 9 wt. %, from about 0.5 to about 8 wt. %, from about 1 to about 7 wt. %, from about 3 to about 6 wt. %, from about 5 to about 10 wt. %, or from about 2 wt. % to about 12 wt. %.

Like the acidic composition, the chlorite composition can optionally include additional additives including diluents, pH adjusters, buffers, surfactants, emollients, moisturizers, film formers, foaming agents, thickeners, dyes, preservatives, additional antimicrobial agents, and mixtures thereof. In some embodiments, it may be desirable to formulate additional additives into the acidic composition and not the chlorite composition to avoid reactions with the chlorite salt. In some embodiments, the chlorite composition consists of the chlorite salt and an optional thickener, pH adjuster, and water or diluent.

The chlorite composition may optionally include a pH adjuster or buffer to bring the pH of the chlorite composition to about 9.0 to about 13, about 10 to about 13, about 11 to about 13, about 9.0 to about 12, about 9.0 to about 11, or about 9.0 to about 10. Exemplary pH adjusters include sodium hydroxide, potassium hydroxide, monoethanolamine, diethanolamine, triethanolamine, sodium carbonate, sodium bicarbonate, potassium carbonate, potassium bicarbonate. Preferred pH adjusters are sodium hydroxide and potassium hydroxide. The chlorite composition may optionally include water as a diluent. Water may be present in the chlorite composition in an amount from about 80 to about 99.99 wt. %, from about 90 to about 99.99 wt. %, or from about 95 to about 99.99 wt. %.

The surfactants, emollients, moisturizers, film formers, foaming agents, thickeners, dyes, preservatives, additional antimicrobial agents, and the like described above for the acidic composition can also be used in the chlorite composition. Which composition includes which additives may be left to a formulator to decide based on preference, the relative concentration of the acidic composition and chlorite composition, or which composition is more desirable for a particular raw material. For example, if a super concentrated chlorite composition is made and added into the acidic composition in a ratio of 1 part chlorite composition to 40 parts acidic composition, it may be desirable to incorporate any additives into the acidic composition. If the acidic composition and the chlorite composition are combined in equal or near equal parts, the formulator may choose to include additives in either composition or in both compositions. Further, the pH stability of a certain additive may indicate that it should be included in the acidic composition or the chlorite composition (having an alkaline pH). A person skilled in the art will be able to determine which additives to include in the acidic composition or the chlorite composition without undue burden.

### Mixing the Acidic Composition and the Chlorite Composition

The acidic composition and the chlorite compositions are normally liquids that are combined together and mixed to form the chlorine dioxide composition. The acidic composition and the chlorite composition can be mixed in a ratio of about 1:4 to about about 100:1 to about 1:2, about 50:1 to about 40:1, or about 1:1 to about 40:1 parts acidic composition to chlorite composition.

The acidic composition and chlorite composition can be mixed manually by pouring, stirring, rocking, shaking, allowing the composition to rest for a period of time and the compositions to mix by dilution, and combinations thereof. The acidic composition and chlorite composition are provided in packaging or a container. The acidic composition and chlorite composition can be added to a third container and mixed in that third container. The third container could be a spray bottle, a bucket, or an application device. Alternatively, the container for the acidic composition or the chlorite composition can be large enough that the dairy farmer could add one composition to the container of the other composition.

In some embodiments, a mixing device can be used to mix the acidic composition and the chlorite composition. An exemplary mixing device includes automated mixing equipment comprising pumps and hoses placed inside of the packaging for the acidic composition and the chlorite composition. The pumps draw product out of the acidic composition and the chlorite composition packages and combine them together in a third container. Another exemplary mixing device would include pumps and hoses but dose the proper amount of acidic composition and chlorite composition by weight (e.g., using a load cell).

In some embodiments, the acidic composition and the chlorite compositions are pre-packaged in containers and sold together with instructions to mix the compositions together. For example, in some embodiments the acidic composition is sold in a 18.9 liter (5 gallon) container at 2.3 wt. % acid and the chlorite composition is sold in a 3.78 liter (1 gallon) container at 3.65 wt. % chlorite with instructions to add 3.5 ounces of the chlorite composition to one liter of the acidic composition and then mix. In some embodiments, the container size for the acidic composition and chlorite composition are selected so that when the acidic composition and chlorite composition are mixed, they are mixed in the proper ratio. For example, the entire container of the chlorite composition can be added into the acidic composition container and then mixed. Exemplary sizes for this embodiment include a 20 liter package for the acidic composition and a 500 ml package of the chlorite composition.

Once mixed, the acidic composition and the chlorite composition are allowed to react for a period of time in order to generate the chlorine dioxide. This reaction time can last from about 5 seconds to about 5 days, from about 10 seconds to about 1 day, from about 30 seconds to about 30 minutes, from about 30 seconds to about 5 minutes, and from about 30 seconds to about 1 minute.

### Using the Chlorine Dioxide Composition

The resulting chlorine dioxide composition is used on animal skin and animal teats to prevent or reduce mastitis infections. The chlorine dioxide composition can be applied to the animal skin by washing, wiping, sponging, misting, foaming, dipping, spraying, flooding, or a combination of these. In some embodiments, the chlorine dioxide composition is used before milking as a pre-milking composition in the form of a wash, spray, foam, wipe, or dip. Such pre-milking compositions are useful to reduce microorganisms that are on the skin as a result of the animal walking around the field or barn. Pre-milking compositions are useful in removing dirt, debris, manure, and microorganisms that could cause mastitis if allowed to infect the mammary gland of the animal. Removing such dirt, debris, manure, and microorganisms before milking also means that the milking equipment and resulting milk is not contaminated with dirt, debris, manure, or microorganisms. Using a pre-milking composition renders the animal skin and teat clean before milking, which helps keep the milking equipment and milk clean.

Pre-milking compositions are generally formulated to run off, wash off, or be wiped off the animal skin before milking. Accordingly, in some embodiments, the pre-milking compositions have a water-like viscosity, or a viscosity of about 0 mPas to about 200 mPas, about 10 mPas to about 100 mPas, or about 10 mPas to about 50 mPas when measured with a Brookfield viscometer, spindle 2 at 12 rpm at 20° C. In some embodiments, the pre-milking compositions are free of thickeners or film forming agents.

In some embodiments, the pre-milking compositions are generated as foaming compositions. In some embodiments, foaming may be generated manually (e.g., by using a foaming dip cup) or automatically by using an applicator such as the Teat Foamer sold by Lafferty or the Power Foamer sold by Ambic. Suitable foaming generates a foam that is capable of clinging to the vertical portion of animal teats and remains as a foam for a period of time needed to provide the antimicrobial effect.

In some embodiments, the chlorine dioxide compositions are formulated as post-milking compositions that are applied to the animal skin or teat after milking. The post-milking compositions are beneficial to reduce any microorganisms that the animal encountered during the milking process, for example, if the milking equipment was contaminated by another animal in the animal population, e.g., the heard or the flock. This helps prevent the spread of microorganisms within the animal population.

The post-milking compositions can be formulated as a wash, spray, foam, or dip. In some embodiments, the post-milking compositions are formulated with a thickener or a film forming polymer to help the composition stay on the animal skin after milking. This provides an ongoing antimicrobial benefit if the animal encounters microorganisms in the field or the barn as it walks around. When the composition is formulated with emollients, moisturizers, or humectants, having the composition stay on the skin provides an ongoing skin health benefit in that it allows those emollients, moisturizers, or humectants to continue to penetrate into the skin. The film formed by the film forming agent also provides a physical barrier for protection against cold temperatures, wind, dirt, or microorganisms. When the post-milking compositions are formulated to be film forming, the film stays on the skin for about 1 minutes to about 12 hours, about 3 minutes to about 8 hours, or about 5 minutes to about 4 hours.

In some embodiments, post-milking compositions are formulated to cling to the skin. Accordingly, in some embodiments, the post-milking compositions have a viscosity that promotes vertical cling of from about 10 mPas to 3000 mPas, from about mPas to about 2500 mPas, or about 300 mPas to about 2000 mPas when measured with a Brookfield viscometer spindle 2 with 12 rpm at 20° C.

In some embodiments, the pre- or post-milking compositions include a dye or a foaming agent. The dye or foam is beneficial as an indicator for which animals have had the composition applied already, ensuring that the application covers the entire teat area, and, in the case of post-milking compositions, the dye serves as a visual indicator for which animals have been milked and, conversely, the absence of the dye serves as a visual indicator for which animals have not been milked.

### EXAMPLES

### Example 1

Example 1 measured the chlorine dioxide generation curves of acetic acid, citric acid, lactic acid, and glycolic acid in the absence of an aldehyde. For this experiment, 5 an acidic composition with various acids and acid concentrations was mixed with a chlorite composition in various concentrations. The resulting chlorine dioxide generation was measured at 15 minutes, 1 hour, 1 day, 3 days, 7 days, 14 days, and 21 days after the compositions were combined. The results are found in Table 1.

TABLE 1
Chlorine Dioxide Generation Curves for Various Organic Acids

Acetic Acid

| | | |
|---|---|---|
| Acid | Chlorite | |
| concentration | concentration | Chlorine dioxide concentration |

| | A: Acetic.Acid | B: Chlorite | 15 min | 1 hr | 1 day | 3 day | 7 day | 14 day | 21 day |
|---|---|---|---|---|---|---|---|---|---|
| Run | % w/w | % w/w | ppm | ppm | ppm | ppm | ppm | ppm | ppm |
| 1 | 0.5 | 0.4 | 22 | 23 | 168 | 380.16 | 431 | 541 | 636 |
| 2 | 1 | 1 | 48 | 69.05 | 556 | 895.22 | 1311 | 1667 | 2000 |
| 3 | 4.5 | 0.2 | 83.2 | 117 | 292.18 | 416.83 | 526 | 599.64 | 591.28 |
| 4 | 2.2 | 0.5 | 78.5 | 105.28 | 471.39 | 767.57 | 977.11 | 1240.41 | 1302.28 |
| 5 | 4.5 | 1 | 179.5 | 587.12 | 1532.47 | 2101.96 | 2499.03 | 2893.45 | 2912.12 |
| 6 | 2.2 | 0.5 | 65.9 | 101.7 | 471.2 | 795.04 | 971.16 | 1232.4 | 1305.85 |
| 7 | 2.2 | 0.5 | 72.19 | 98 | 471.72 | 779.59 | 1022.87 | 1218.71 | 1300.07 |
| 8 | 2 | 0.1 | 14.53 | 32 | 102.87 | 178.91 | 196 | 252.02 | 253.32 |
| | Citric Acid | | | | | | | | |

| Acid | Chlorite | |
|---|---|---|
| concentration | concentration | Chlorine dioxide concentration |

| | A: Citric.Acid | B: Chlorite | 15 min | 1 hr | 1 day | 3 day | 7 day | 14 day | 21 day |
|---|---|---|---|---|---|---|---|---|---|
| Run | % w/w | % w/w | ppm | ppm | ppm | ppm | ppm | ppm | ppm |
| 1 | 4 | 1 | 911 | 1307.17 | 2915.14 | 3320.64 | 3167.09 | 3060.44 | 2945.94 |
| 2 | 2.2 | 0.5 | 397.03 | 525.37 | 1268.05 | 1562.67 | 1746.1 | 1752.84 | 1694.62 |
| 3 | 4.5 | 0.4 | 544.63 | 664.2 | 1203.46 | 1424.68 | 1444.88 | 1393.41 | 1297.78 |
| 4 | 3.5 | 0.1 | 173.3 | 163.43 | 255.99 | 392.07 | 322 | 323.06 | 304.87 |
| 5 | 0.5 | 1 | 127.18 | 204 | 888 | 1168.61 | 1524 | 1799 | 1870 |
| 6 | 0.5 | 0.2 | 61.35 | 68.22 | 243 | 363.81 | 480 | 570 | 621 |
| 7 | 2.2 | 0.5 | 426.65 | 534.41 | 1252.15 | 1589.24 | 1752.59 | 1743.39 | 1708.7 |
| 8 | 2.2 | 0.5 | 398.07 | 512.38 | 1236.91 | 1537.57 | 1733.25 | 1749.87 | 1705.12 |

| | Glycolic Acid | | | | | | | | |
|---|---|---|---|---|---|---|---|---|---|
| | Acid | Chlorite | | | | | | | |
| | concentration | concentration | Chlorine dioxide concentration | | | | | | |
| | A: Glycolic.Acid | B: Chlorite | 15 min | 1 hr | 1 day | 3 day | 7 day | 14 day | 21 day |
| Run | % w/w | % w/w | ppm | ppm | ppm | ppm | ppm | ppm | ppm |
| 1 | 0.5 | 1 | 119 | 183.97 | 927 | 1270.08 | 1680 | 1910 | 1960 |
| 2 | 1.5 | 1 | 291.77 | 507.99 | 1760.78 | 2277.51 | 2586.34 | 2589.24 | 2482.65 |
| 3 | 2.2 | 0.5 | 422 | 452.12 | 1126.15 | 1371.09 | 1448.6 | 1432.34 | 1186.05 |
| 4 | 1 | 0.1 | 74 | 85 | 158 | 205.9 | 257 | 298 | 282 |
| 5 | 2.2 | 0.5 | 318.93 | 454.2 | 1067.52 | 1396.7 | 1445.1 | 1401.97 | 1355.59 |
| 6 | 3.5 | 1 | 797 | 1117.1 | 2531.95 | 2874.64 | 2859.03 | 2684.46 | 2204.4 |
| 7 | 2.2 | 0.5 | 421 | 508.4 | 1064.87 | 1376.93 | 1452.27 | 1439.82 | 1363.05 |
| 8 | 3.5 | 0.1 | 196.23 | 194.25 | 248.03 | 289.57 | 305 | 307.09 | 278.64 |

| | |
|---|---|
| Lactic Acid | |
| Acid | Chlorite |

| | | |
|---|---|---|
| concentration | concentration | Chlorine dioxide concentration |

| | A: Lactic.Acid | B: Chlorite | 15 min | 1 hr | 1 day | 3 day | 7 day | 14 day | 21 day |
|---|---|---|---|---|---|---|---|---|---|
| Run | % w/w | % w/w | ppm | ppm | ppm | ppm | ppm | ppm | ppm |
| 1 | 2.2 | 0.5 | 276 | 341.27 | 974 | 1263 | 1356.05 | 1093 | 601.86 |
| 2 | 4.5 | 0.4 | 414 | 498.02 | 1033.72 | 1147 | 951.6 | 326 | 105.89 |
| 3 | 3.5 | 0.1 | 152 | 148.02 | 210.81 | 264.35 | 282 | 170.13 | 68.41 |
| 4 | 0.5 | 0.2 | 37 | 40.6 | 175 | 271.48 | 386 | 454 | 471 |
| 5 | 2.2 | 0.5 | 266.26 | 341.12 | 972.08 | 1265 | 1404.08 | 1115 | 603.05 |
| 6 | 0.5 | 1 | 84 | 134.33 | 771 | 1097.95 | 1522 | 1746 | 1755 |
| 7 | 2.2 | 0.5 | 282.47 | 347.7 | 992 | 1249 | 1363.21 | 1111 | 614.9 |
| 8 | 4 | 1 | 714 | 1049.13 | 2463.41 | 2844 | 2218.94 | 744 | 266.55 |

The results in Table 1 show that the acids were able to generate sufficient amounts of chlorine dioxide but the quantity and the rate of chlorine dioxide generation varied depending on the acid and the relative concentrations of acid and chlorite.

### Example 2

Example 2 tested effect of raw material storage on the resulting chlorine dioxide generation. The following formulas were generated:

| | |
|---|---|
| Acid Formula | Chlorite Formula |

| | | | |
|---|---|---|---|
| Deionized Water | 1748.40 | Deionized Water | 3926.86 |
| Dye | 2.01 | Sodium Chlorite | 68.02 |
| | | Solution (25%) | |
| Citric Acid | 20.00 | KOH (50%) | 3.41 |
| (anhydrous) | | | |
| Glycerine (99.5%) | 100.68 | | |
| Sorbitol (70%) | 100.88 | | |
| Glyoxylic Acid | 2.01 | | |
| (50%) | | | |
| KOH (50%) | 1.78 | | |
| Alkyl Polyglucoside | 26.00 | | |
| Adjusted pH | 2.49 | | 12.01 |

The acid formula and chlorite formula were tested using fresh formulas and after storage of the raw materials at 54° C. for two weeks. For testing, the acid and chlorite formulas were mixed together in a ratio of 1:1. Chlorine dioxide (ClO₂) and acidified sodium chlorite (NaClO₂) were measured after mixing at 5 minutes, 1 day, 2 days, 7 days, 14 days, 21 days, and 30 days. The results are shown in Table 2.

TABLE 2
Chlorine Dioxide and Acidified Sodium Chlorite
Generation in Fresh and Aged Reagents

| | Acid | | | |
|---|---|---|---|---|
| | Age | | fresh | 2 w@54° C. |
| | Chlorite | | | |
| | Age | | fresh | 2 w@54° C. |
| ClO2 | 5 | min | 392.94 | 405.65 |
| (ppm) | 1 | d | 464.14 | 485.12 |
| | 2 | d | 476.79 | 513.91 |
| | 7 | d | 494.8 | 529.73 |
| | 14 | d | 454.19 | 512.27 |
| | 21 | d | 465.86 | 497.67 |
| | 30 | d | 437 | 497 |
| NaClO2 | 5 | min | 1258 | 1251 |
| (ppm) | 1 | d | 1022 | 994 |
| | 2 | d | 1005 | 872 |
| | 7 | d | 869 | 686 |
| | 14 | d | 761 | 529 |
| | 21 | d | 487 | 419 |
| | 30 | d | 351 | 290 |

The results in Table 2 show that the starting acidic and chlorite compositions can be stored for up to two years without losing the ability to generate chlorine dioxide.

### Example 3

Example 3 tested the antimicrobial efficacy of various compositions against yeast (Candida albicans) and bacteria (S. aureus) under pre-milking conditions. The formulas in Table 3 were prepared and mixed in a 1:1 ratio. The mixed chlorine dioxide composition was then tested under the conditions in the table using the regulatory test methods EN1656 and EN1657. Table 3 shows the log reduction for pre-milking formulas.

TABLE 3
Pre-Milking Teat Dip Formulas

| | | | | | | |
|---|---|---|---|---|---|---|
| 1 | 2 | 3 | 4 | 5 | 6 | 7 |

Component 1
(Acidic
Composition)

Ratio 1:1 (w/w) for Component 1 and 2
Efficacy: log 5 for bacteria and log 4 for yeast

| | | | | | | | |
|---|---|---|---|---|---|---|---|
| Water Deionized | 78.09 | 84.11 | 85.11 | 83.17 | 88.46 | 87.60 | 87.60 |
| Glycerine | 5.00 | 5.00 | 5.00 | 5.00 | 5.00 | 5.00 | 5.00 |
| Sorbitol | 5.00 | 5.00 | 5.00 | 5.00 | 5.00 | 5.00 | 5.00 |
| APG | | 1.00 | | 2.00 | 0.50 | 1.30 | 1.30 |
| Linear-Alkyl- | 1.00 | | | | | | |
| Sulfate | | | | | | | |
| Citric Acid anyhdr. | 9.90 | 4.50 | 4.50 | 4.50 | 1.00 | 1.00 | 1.00 |
| KOH/50% | 0.91 | 0.29 | 0.29 | 0.23 | n.a. | 0.07 | 0.07 |
| Anisaldehyde | v | | | | 0.04 | | |
| Phenylacetaldehyde | | | | 0.10 | | | |
| Benzaladehyde | 0.10 | 0.10 | 0.10 | | | | |
| Glyoxylic Acid/ | | | | | | 0.03 | 0.03 |
| 50% | | | | | | | |
| pH | 2.10 | 2.10 | 2.10 | 2.10 | 2.20 | 2.50 | 2.50 |
| Component 2 | | | | | | | |
| (Chlorite | | | | | | | |
| Composition) | | | | | | | |
| Water Deionized | 97.60 | 98.40 | 98.40 | 98.00 | 98.80 | 98.81 | 98.66 |
| KOH/50% | | | | | | 0.09 | 0.09 |
| Sodium Chlorite | 2.40 | 1.60 | 1.60 | 2.00 | 1.20 | 1.10 | 1.25 |
| Solution 25% | | | | | | | |
| pH | / | / | / | / | / | 12.00 | 12.00 |

Efficacy Testing

| time after mixing | 15 | min | 15 | min | 15 | min | 5 | min | 5 | min | 5 | min | 5 | min |
|---|---|---|---|---|---|---|---|---|---|---|---|---|---|---|
| dilution/% | 80 | | 80 | | 80 | | 80 | | 80 | | 80 | | 80 | |
| contact time | 60 | s | 60 | s | 60 | s | 60 | s | 60 | s | 60 | s | 60 | s |

| Bacteria | pass | pass | | pass | | pass | | pass | | fail | | fail | |
|---|---|---|---|---|---|---|---|---|---|---|---|---|---|
| Yeast | pass | pass | | pass | | pass | | pass | | fail | | pass | |
| time after mixing | | 15 | min | 15 | min | 24 | h | 5 | min | 5 | min | 5 | min |
| dilution/% | | 40 | | 40 | | 80 | | 40 | | 80 | | 80 | |
| contact time | | 60 | s | 60 | s | 60 | s | 60 | s | 30 | s | 30 | s |
| Bacteria | | fail | | fail | | pass | | fail | | fail | | fail | |
| Yeast | | pass | | fail | | pass | | fail | | fail | | pass | |
| time after mixing | | | | | | 48 | h | | | | | | |
| dilution/% | | | | | | 80 | | | | | | | |
| contact time | | | | | | 60 | s | | | | | | |

| | |
|---|---|
| Bacteria | pass |
| Yeast | pass |

| | | | | | |
|---|---|---|---|---|---|
| 8 | 9 | 10 | 11 | 12 | 13 |

Component 1
(Acidic
Composition)

Ratio 1:1 (w/w) for Component 1 and 2
Efficacy: log 5 for bacteria and log 4 for yeast

| | | | | | | |
|---|---|---|---|---|---|---|
| Water Deionized | 87.58 | 87.58 | 87.58 | 87.58 | 87.58 | 87.52 |
| Glycerine | 5.00 | 5.00 | 5.00 | 5.00 | 5.00 | 5.00 |
| Sorbitol | 5.00 | 5.00 | 5.00 | 5.00 | 5.00 | 5.00 |
| APG | 1.30 | 1.30 | 1.30 | 1.30 | 1.30 | 1.30 |
| Linear-Alkyl- | | | | | | |
| Sulfate | | | | | | |
| Citric Acid anyhdr. | 1.00 | 1.00 | 1.00 | 1.00 | 1.00 | 1.00 |
| KOH/50% | 0.07 | 0.07 | 0.07 | 0.07 | 0.07 | 0.08 |
| Anisaldehyde | | | | | | |
| Phenylacetaldehyde | | | | | | |
| Benzaladehyde | | | | | | |
| Glyoxylic Acid/ | 0.05 | 0.05 | 0.05 | 0.06 | 0.06 | 0.10 |
| 50% | | | | | | |
| pH | 2.50 | 2.50 | 2.50 | 2.50 | 2.50 | 2.50 |
| Component 2 | | | | | | |
| (Chlorite | | | | | | |
| Composition) | | | | | | |
| Water Deionized | 99.02 | 98.66 | 98.68 | 98.49 | 98.40 | 98.17 |
| KOH/50% | 0.08 | 0.09 | 0.08 | 0.09 | 0.10 | 0.13 |
| Sodium Chlorite | 0.90 | 1.25 | 1.25 | 1.42 | 1.50 | 1.70 |
| Solution 25% | | | | | | |
| pH | 12.00 | 12.00 | 12.00 | 12.00 | 12.00 | 12.00 |

Efficacy Testing

| time after mixing | 5 | min | 5 | min | 5 | min | 14 | d | 5 | min | 5 | min | 5 min | |
|---|---|---|---|---|---|---|---|---|---|---|---|---|---|---|
| dilution/% | 80 | | 80 | | 80 | | 80 | | 80 | | 80 | | 80 | |
| contact time | 60 | s | 60 | s | 30 | s | 30 | s | 30 | s | 30 | s | 30 | s |
| Bacteria | fail | | pass | | fail | | pass | | fail | | fail | | pass | |
| Yeast | pass | | pass | | pass | | pass | | fail | | fail | | pass | |
| time after mixing | 5 | min | 5 | min | 48 | h | 21 | d | 21 | d | 21 | d | 5 | min |
| dilution/% | 80 | | 80 | | 80 | | 80 | | 80 | | 80 | | 80 | |
| contact time | 30 | s | 30 | s | 30 | s | 30 | s | 30 | s | 30 | s | 60 | s |
| Bacteria | fail | | pass | | pass | | fail | | pass | | fail | | pass | |
| Yeast | pass | | pass | | pass | | pass | | fail | | pass | | pass | |
| time after mixing | | | | | 7 | d | 30 | d | 30 | d | 30 | d | | |
| dilution/% | | | | | 80 | | 80 | | 80 | | 80 | | | |
| contact time | | | | | 30 | s | 30 | s | 30 | s | 30 | s | | |

| | | | | |
|---|---|---|---|---|
| Bacteria | pass | fail | pass | pass |
| Yeast | pass | pass | fail | pass |

### Example 4

Example 4 tested the antimicrobial efficacy of various compositions against yeast (Candida albicans) and bacteria (S. aureus) under post-milking conditions. The formulas in Table 4 were prepared and mixed in a 1:1 ratio. The mixed chlorine dioxide composition was then tested under the conditions in the table using the regulatory test methods EN1656 and EN1657. Table 4 shows the log reduction for post-milking formulas.

TABLE 4
Post-Milking Teat Dip Formulas

| | 14 | 15 | 16 | 17 | 18 | 19 | 20 | 21 | 22 |
|---|---|---|---|---|---|---|---|---|---|
| Component 1 | | | | | | | | | |

Ratio 1:1 (w/w) for Component 1 and 2
Efficacy: log 5 for bacteria and log 4 for yeast

| | | | | | | | | | |
|---|---|---|---|---|---|---|---|---|---|
| Water Deionized | 88.00 | 87.52 | 88.46 | 88.68 | 88.75 | 88.32 | 88.38 | 88.28 | 88.58 |
| Xanthan Gum | | | | | | 0.30 | 0.30 | 0.30 | |
| Glycerine | 5.00 | 5.00 | 5.00 | 5.00 | 5.00 | 5.00 | 5.00 | 5.00 | 5.00 |
| Sorbitol | 5.00 | 5.00 | 5.00 | 5.00 | 5.00 | 5.00 | 5.00 | 5.00 | 5.00 |
| Phthalocyanine dye | | | | | | | | 0.10 | 0.10 |
| APG | 0.50 | 0.50 | 0.50 | | | | | | |
| Linear-Alkyl-Sulfate | | | | 0.20 | 0.20 | 0.20 | 0.20 | 0.20 | 0.20 |
| Citric Acid anyhdr. | 1.00 | 1.00 | 1.00 | 1.00 | 1.00 | 1.00 | 1.00 | 1.00 | 1.00 |
| KOH/50% | 0.46 | 0.91 | | 0.07 | | 0.13 | 0.07 | 0.07 | 0.07 |
| Anisaldehyde | | | 0.04 | | | | | | |
| Benzaladehyde | 0.04 | | | | | | | | |
| Glyoxylic Acid/50% | | 0.07 | | 0.05 | 0.05 | 0.05 | 0.05 | 0.05 | 0.05 |
| Viscosity [mPas], SPDL2, 12 rpm | | | | | | 676 | 676 | | |
| pH | 3.50 | 4.50 | 2.20 | 2.50 | 2.50 | 2.66 | 2.66 | 2.50 | 2.50 |
| Component 2 | | | | | | | | | |
| Water Deionized | 98.80 | 98.00 | 98.80 | 99.02 | 99.15 | 98.38 | 98.85 | 98.73 | 99.03 |
| KOH/50% | | | n.a. | 0.08 | 0.10 | 0.08 | 0.10 | 0.07 | 0.07 |
| Xanthan Gum | | | | | | 0.30 | 0.30 | 0.30 | |
| Sodium Chlorite Solution 25% | 1.20 | 2.00 | 1.20 | 0.90 | 0.75 | 1.25 | 0.75 | 0.90 | 0.90 |
| pH | / | / | n.a. | 12.00 | 12.00 | 11.92 | 11.84 | 12.00 | 12.00 |

(11.2)

Efficacy Testing

| time after mixing | 5 min | 5 min | 5 min | 5 min | 5 min | 5 min | 5 min | 5 | min | 5 | min |
|---|---|---|---|---|---|---|---|---|---|---|---|
| dilution/% | 80 | 80 | 80 | 80 | 80 | 80 | 80 | 80 | | 80 | |
| contact time | 5 min | 5 min | 5 min | 5 min | 5 min | 5 min | 5 min | 5 | min | 5 | min |
| Bacteria | pass | pass | pass | pass | pass | pass | pass | pass | | pass | |
| Yeast | pass | pass | pass | pass | fail | pass | pass | pass | | pass | |
| time after mixing | 5 min | 5 min | 5 min | 5 min | 5 min | 5 min | 5 min | 30 | d | 30 | d |
| dilution/% | 40 | 40 | 40 | 40 | 40 | 40 | 40 | 80 | | 80 | |
| contact time | 5 min | 5 min | 5 min | 5 min | 5 min | 5 min | 5 min | 5 | min | 5 | min |

| | | | | | | | | | |
|---|---|---|---|---|---|---|---|---|---|
| Bacteria | pass | pass | pass | pass | pass | pass | pass | pass | pass |
| Yeast | pass | pass | pass | pass | fail | pass | pass | pass | pass |

### Example 5

Example 5 tested the effect of a pre-milking composition on animal skin. For this example, dairy farmers were asked to score the effect of a pre-milking composition of the present disclosure on the skin health of cows. Farmers from four farms were asked to apply the compositions as a foam before milking for one month. The farmers has previously been using a lactic acid/hydrogen peroxide-based ready-to-use teat dip composition. The farmers scored the animal teat skin before starting the test and after one month of applying the composition using the following scale:
0—damage to barrel not from dip
1—soft, smooth skin
2—dryness of skin
3—sloughing of skin, cracks, peeling
4—cracking and/or bleeding on the barrel

TABLE 5

| | Skin Score Before | Skin Score After | |
|---|---|---|---|
| | the Test | the Test | % Improvement |
| Farm 1 | 1.35 | 1.16 | 14% |
| Farm 2 | 1.23 | 1.14 | 7% |
| Farm 3 | 1.70 | 1.17 | 31% |
| Farm 4 | 1.82 | 1.15 | 37% |

In addition to the skin scoring, the farmers also noted that the pre-milking composition had good foam coverage, good visibility, good dirt penetration, it was easy to wipe dirt and foam from the teats, the smooth teat skin was easier to clean, the cows were entering the milking room with cleaner teats, and the cows were more relaxed when entering the milking room.

The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A method of generating a chlorine dioxide composition comprising mixing an acidic composition and a chlorite composition together to form a chlorine dioxide composition, the acidic composition comprising an acid and an aldehyde, the chlorite composition comprising a chlorite salt, and the chlorine dioxide composition comprising chlorine dioxide,
wherein the chlorine dioxide composition comprises a quantity of the chlorite salt and the aldehyde that are unreacted, and
wherein the reaction between the chlorite salt and the aldehyde continues to generate chlorine dioxide composition for at least seven days after combining the acidic composition and the chlorite composition.

2. The method of claim 1, wherein the acid is selected from the group consisting of acetic acid, citric acid, lactic acid, glycolic acid, and mixtures thereof.

3. The method of claim 1, wherein the acid is present in the acidic composition in an amount from about 0.1 to about 10 wt. %.

4. The method of claim 1, wherein the aldehyde is selected from the group consisting of glutaraldehyde, formaldehyde, glyoxylic acid, glyoxal, succinaldehyde, adipaldehyde, and mixtures thereof.

5. The method of claim 1, wherein the aldehyde is present in the acidic composition in an amount from about 50 ppm to about 3000 ppm.

6. The method of claim 1, wherein the acidic composition has a pH of about 2 to about 7.

7. The method of claim 1, wherein the acidic composition or chlorite composition further comprises an additional additive selected from the group consisting of diluents, pH adjusters, buffers, surfactants, emollients, moisturizers, film formers, foaming agents, thickeners, dyes, preservatives, additional antimicrobial agents, and mixtures thereof.

8. The method of claim 1, wherein the chlorite salt is selected from the group consisting of sodium chlorite, potassium chlorite, and mixtures thereof.

9. The method of claim 1, wherein the chlorite salt is present in the chlorite composition an amount of about 0.04 to about 12.5 wt. %.

10. The method of claim 1, wherein the chlorite composition further comprises an additive selected from the group consisting of diluents, pH adjusters, buffers, surfactants, emollients, moisturizers, film formers, foaming agents, thickeners, dyes, preservatives, additional antimicrobial agents, and mixtures thereof.

11. The method of claim 1, wherein the chlorite composition has a pH of about 9 to about 13.

12. The method of claim 1, wherein the chlorine dioxide is present in the chlorine dioxide composition in an amount of about 50 ppm to about 3000 ppm.

13. The method of claim 1, wherein the chlorine dioxide composition has a pH of about 2 to about 7.

14. The method of claim 1, wherein the acidic composition and the chlorite composition are mixed together in a ratio of about 1:4 to about 40:1.

15. The method of claim 1, wherein the acidic composition and the chlorite composition are mixed together in a ratio of about 100:1 to about 1:2.

16. The method of claim 1, wherein the acidic composition and the chlorite composition are mixed together in a ratio of about 50:1 to about 40:1.

17. The method of claim 1, wherein the chlorine dioxide concentration in the chlorine dioxide composition is greater than 50 ppm within five minutes of when the acidic composition and the chlorite composition are first mixed together

18. The method of claim 1, wherein the chlorine dioxide composition has at least a 3-log reduction against E. coli within 5 minutes at room temperature.

19. The method of claim 1, wherein the concentration of the aldehyde in the chlorine dioxide composition is less than 10 ppm 1 minute after the acidic composition and the chlorite composition are mixed together.

20. The method of claim 1, wherein the chlorine dioxide composition has a viscosity of about 0 to about 200 mPas when measured with a Brookfield viscometer using a 2 spindle at 12 rpm and 20° C.

21. A method of reducing microorganisms on animal skin comprising:
(a) mixing an acidic composition and a chlorite composition together to form a chlorine dioxide composition,
the acidic composition comprising an acid and an aldehyde;
the chlorite composition comprising a chlorite salt; and
the chlorine dioxide composition comprising chlorine dioxide; and
(b) applying the chlorine dioxide composition to animal skin
wherein the chlorine dioxide composition comprises a quantity of the chlorite salt and the aldehyde that are unreacted, and
wherein the reaction between the chlorite salt and the aldehyde continues to generate chlorine dioxide composition for at least seven days after combining the acidic composition and the chlorite composition.

22. The method of claim 21, wherein the acid is selected from the group consisting of acetic acid, citric acid, lactic acid, glycolic acid, and mixtures thereof.

23. The method of claim 21, wherein the acid is present in the acidic composition in an amount from about 0.1 wt. % to about 10 wt. %.

24. The method of claim 21, wherein the aldehyde is selected from the group consisting of glutaraldehyde, formaldehyde, glyoxylic acid, glyoxal, succinaldehyde, adipaldehyde, and mixtures thereof.

25. The method of claim 21, wherein the aldehyde is present in the acidic composition in an amount from about 50 ppm to about 3000 ppm.

26. The method of claim 21, wherein the acidic composition has a pH of about 2 to about 7.

27. The method of claim 21, wherein the acidic composition or chlorite composition further comprises an additional additive selected from the group consisting of diluents, pH adjusters, buffers, surfactants, emollients, moisturizers, film formers, foaming agents, thickeners, dyes, preservatives, additional antimicrobial agents, and mixtures thereof.

28. The method of claim 21, wherein the chlorite salt is selected from the group of sodium chlorite, potassium chlorite, and mixtures thereof.

29. The method of claim 21, wherein the chlorite salt is present in the chlorite composition in an amount of about 0.04 to about 12.5 wt. %.

30. The method of claim 21, wherein the chlorite composition has a pH of about 9 to about 13.

31. The method of claim 21, wherein the chlorine dioxide is present in the chlorine dioxide composition in an amount of about 50 ppm to about 3000 ppm.

32. The method of claim 21, wherein the chlorine dioxide composition has a pH of about 2 to about 7.

33. The method of claim 21, wherein the acidic composition and the chlorite composition are mixed together in a ratio of about 1:4 to about 40:1.

34. The method of claim 21, wherein the acidic composition and the chlorite composition are mixed together in a ratio of about 100:1 to about 1:2.

35. The method of claim 21, wherein the acidic composition and the chlorite composition are mixed together in a ratio of about 50:1 to about 40:1.

36. The method of claim 21, wherein the chlorine dioxide concentration in the chlorine dioxide composition is greater than 50 ppm within five minutes of when the acidic composition and the chlorite composition are first mixed together

37. The method of claim 21, wherein the chlorine dioxide composition has at least a 3-log reduction against E. coli within 5 minutes at room temperature.

38. The method of claim 21, wherein the concentration of the aldehyde in the chlorine dioxide composition is less than 10 ppm 1 minute after the acidic composition and the chlorite composition are mixed together.

39. The method of claim 21, wherein the chlorine dioxide composition has a viscosity of about 0 to about 200 mPas when measured with a Brookfield viscometer using a spindle 2 at 12 rpm at 20° C.

40. The method of claim 21, wherein the chlorine dioxide composition is applied to the animal skin by washing, spraying, dipping, misting, foaming, or a combination thereof.

41. The method of claim 21, wherein the chlorine dioxide composition is applied to the animal skin before milking.

42. The method of claim 21, wherein the chlorine dioxide composition is applied to the animal skin after milking.

43. The method of claim 21, wherein the chlorine dioxide composition further comprises a film former and remains on the animal skin as a film after milking for a period of time.
